(19) United States
(12) Patent Application Publication
(10) Pub. No.: US 2024/0245315 A1
(43) Pub. Date: Jul. 25, 2024

(54) CONTACTLESS SENSOR-DRIVEN DEVICE, SYSTEM AND METHOD ENABLING CARDIOVASCULAR AND RESPIRATORY ASSESSMENT BASED ON FACE AND HAND IMAGING

(71) Applicant: Norbert Health, Inc., Brooklyn, NY (US)

(72) Inventors: Alexandre G. Winter, Brooklyn, NY (US); Jonathan Platkiewicz, Boulogne-Billancourt (FR); Alexis Bienvenu, Brouzet-les-Ales (FR); Xavier Ding, Bagneux (FR); Claire Robichon, Behlenheim (FR)

(21) Appl. No.: 18/413,749

(22) Filed: Jan. 16, 2024

Related U.S. Application Data
(60) Provisional application No. 63/440,241, filed on Jan. 20, 2023.

(51) Int. Cl. A61B 5/024; A61B 5/00; A61B 5/0205

(52) U.S. Cl. CPC A61B 5/02416; A61B 5/0205; A61B 5/7221; A61B 5/7485

(57) ABSTRACT

Embodiments of the present disclosure relate to contactless sensor-driven devices, systems, and methods for performing remote photoplethysmography. In one embodiment, a method of assessing at least one vital sign of a subject comprises: acquiring a first pleth signal based at least in part on capturing ambient light reflected from at least one region of interest (ROI) on a face of the subject; acquiring a second pleth signal from a palmar side of a hand of the subject; and computing estimates of the at least one vital sign of the subject from the first and/or second pleth signals.

### CROSS-REFERENCE TO RELATED APPLICATION(S)

This application claims the benefit of priority of U.S. Provisional Patent Application No. 63/440,241, filed on Jan. 20, 2023, the disclosure of which is hereby incorporated by reference herein in its entirety.

### BACKGROUND

Blood volume change is a rich physiological signal from which one can derive heart rate, respiratory rate, oxygen saturation, blood pressure, atrial fibrillation detection, and other meaningful measurements. Traditional contactless camera-based methods for estimating blood volume change are typically based solely on a facial analysis. While this can be an effective method for estimating a change in blood volume, the performance in utilizing this method significantly drops for when applied to individuals with darker skin.

The use of hand-based remote photoplethysmography (rPPG) can be beneficial in such cases. As the palm typically has a lower melanin content than the face, imaging of the inner hand can compensate for the lack of light reflection in the face. Although there are a few studies that propose hand-based rPPG for practical purposes, there are no known applications for improving the estimation of a change in blood volume. Accordingly, there is a need for improved approaches.

### SUMMARY

The following presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

One aspect of the present disclosure relates to a method of assessing at least one vital sign of a subject, the method comprising: acquiring a first plethysmograph (hereinafter “pleth”) signal based at least in part on capturing ambient light reflected from at least one region of interest (ROI) on a face of the subject; computing a confidence score for the first pleth signal; acquiring a second pleth signal from a palmar side of a hand of the subject, where the second pleth signal is acquired when the confidence score in the first signal does not satisfy a threshold value; and computing estimates of the at least one vital sign of the subject from one or both of the first and second pleth signals and combining the estimates into a final estimate of the at least one vital sign based on the confidence score.

Another aspect of the present disclosure relates to a method of assessing at least one vital sign of a subject, the method comprising: acquiring a first pleth signal based at least in part on capturing ambient light reflected from at least one region of interest (ROI) on a face of the subject; computing a confidence score for the first pleth signal; acquiring a second pleth signal from a palmar side of a hand of the subject, where the second pleth signal is acquired when the confidence score in the first signal does not satisfy a threshold value; combining the first and second pleth signals into a composite pleth signal based on the confidence score; and estimating the at least one vital sign of the subject from the composite pleth signal.

Another aspect of the present disclosure relates to a method of assessing at least one vital sign of a subject, the method comprising: acquiring a first pleth signal based at least in part on capturing ambient light reflected from at least one region of interest (ROI) on a face of the subject; computing a confidence score for the first pleth signal; determining whether the confidence score satisfies a threshold condition; and responsive to determining that the confidence score fails to satisfy the threshold condition, acquiring a second pleth signal from a palmar side of a hand of the subject.

Another aspect of the present disclosure relates to a method of assessing at least one vital sign of a subject, the method comprising: acquiring a first pleth signal based at least in part on capturing ambient light reflected from at least one region of interest (ROI) on a face of the subject; acquiring a second pleth signal from a palmar side of a hand of the subject; computing a confidence score for the first pleth signal or the second pleth signal; and computing estimates of the at least one vital sign of the subject from the first and second pleth signals and combining the estimates into a final estimate of the at least one vital sign based on the confidence score.

In at least one embodiment, the confidence score is computed based on one or more of ambient light, movement of the subject, and regularity of the first pleth signal.

In at least one embodiment, the method further comprises: computing a pulse transmit time based at least in part on the first pleth signal and the second pleth signal.

In at least one embodiment, the subject is prompted to position the palmar side of the hand for acquisition of the second pleth signal.

In at least one embodiment, the method further comprises determining a second confidence score in the second pleth signal. In at least one embodiment, the first pleth signal and the second pleth signal or their respective estimates are weighted by their respective confidence scores.

In at least one embodiment, the first pleth signal is derived from sub-signals collected from a plurality of ROIs on the face of the subject.

In at least one embodiment, the second pleth signal is derived from sub-signals collected from a plurality of ROIs on the hand of the subject.

In at least one embodiment, the first and second pleth signals are derived from image frames collected using a visible light camera and an infrared camera, the infrared camera configured to extract data associated with skin areas inside ROIs identified on image frames collected by the visible light camera.

In at least one embodiment, the vital sign of the subject assessed is a heart rate, a variability in hearth rate, a respiratory rate, an oxygen saturation, a blood pressure or a combination thereof.

Another aspect of the present disclosure relates to a device to perform remote photoplethysmography on a subject (e.g., a human subject), the device comprising: a camera; and a processing device communicatively coupled to camera, where the processing device is configured to perform any of the methods of the preceding embodiments.

Another aspect of the present disclosure relates to a system comprising: a memory; and a processing device communicatively coupled to the memory, where the processing device is configured to perform any of the methods of the preceding embodiments.

Another aspect of the present disclosure relates to a non-transitory computer-readable medium having instructions stored thereon, which, when executed by a processing device, cause the processing device to perform any of the methods of the preceding embodiments.

### BRIEF DESCRIPTION OF DRAWINGS

The examples described herein will be understood more fully from the detailed description given below and from the accompanying drawings, which, however, should not be taken to limit the application to the specific examples, but are for explanation and understanding only.

FIG. 1 is a block diagram illustrating an exemplary system architecture, in accordance with at least one embodiment.

FIG. 2 illustrates a high level overview of the rPPG measurement performed by an exemplary device, in accordance with at least one embodiment.

FIG. 3A illustrates a visible light camera image captured in accordance with at least one embodiment.

FIG. 3B illustrates an infrared camera image captured in accordance with at least one embodiment.

FIG. 3C illustrates detection of a subject's hand and face in a visible light camera image, in accordance with at least one embodiment.

FIG. 3D illustrates detection of the subject's hand and face in an infrared camera image, in accordance with at least one embodiment.

FIG. 3E illustrates combining visible light and infrared images into a composite image, in accordance with at least one embodiment.

FIG. 4 illustrates generating of a composite plethysmograph signal computed from signals derived from the subject's hand and face, in accordance with at least one embodiment.

FIG. 5 is a block diagram illustrating a computer system according to certain embodiments.

### DETAILED DESCRIPTION

The embodiments described herein relate generally to rPPG measurements for determining vital signs and, more specifically, to a non-invasive device, system, and method of performing rPPG using one or more contactless sensors to measure both the face and hand(s) of a subject.

Blood volume change is a rich physiological signal, from which one can derive or detect heart rate, respiratory rate, oxygen saturation, blood pressure, atrial fibrillation detection, and other physiological variables or conditions. Current contactless camera-based methods for estimating blood volume change are based on a facial analysis, where the subject's face is detected, extracted, and its associated pixels are processed. However, the performance of such techniques significantly drops when measurements are performed on the faces of darker-skin subjects. This is essentially due to the lower intensity of the reflected light, which decreases the signal-to-noise ratio.

Certain embodiments of the present disclosure utilize hand-based rPPG to address these limitations by combining face and hand plethysmograph (“pleth”) signals. The palm generally has a lower melanin content than the face, and as such imaging the inner hand can compensate for the lack of light reflection, thus providing a more reliable pleth signal regardless of the subject's skin tone. In other cases when the facial pleth signal is unreliable due to substantial occlusion (e.g., the presence of a head scarf), the hand pleth signal can significantly improve the estimation, or even be substituted for the facial signal. Moreover, this approach can provide detailed information that leverages the difference in dynamics imposed by the physiology of the cardiovascular system. For example, certain embodiments may be utilized to compute blood volume change, as well as compute pulse transit time for accurately measuring blood pressure and pulse wave velocity.

In at least one embodiment, a monitoring device comprising a plurality of contactless sensors is provided. The sensors may be configured to detect, measure, monitor, and provide alerts where appropriate, in connection with various vital signs, movement and recognized routines of an individual. Data collected by sensors may be processed in accordance with various algorithmic processes suited for performing rPPG. These algorithmic processes may be executed directly on the monitoring device, remote from the monitoring device or, in some instances, a combination of both. Additionally, the foregoing sensors may be integrated in a single monitoring device unit, optionally coupled to a centralized monitoring device or any suitable combination thereof depending on the monitoring environment.

In at least one embodiment, the monitoring device may be integrated as part of an overall health monitoring system, where the system may be comprised of the monitoring device, a plurality of mobile devices associated with various individuals (e.g., patients, health care providers, family members, etc.) configured with monitoring/notifying applications (e.g., a caregiver application), one or more servers, one or more databases, and one or more secondary systems that are communicatively coupled to enable the health monitoring system described herein. Such monitoring devices, systems, and methods are disclosed in U.S. patent application Ser. No. 17/225,313, the disclosure of which is hereby incorporated by reference herein in its entirety.

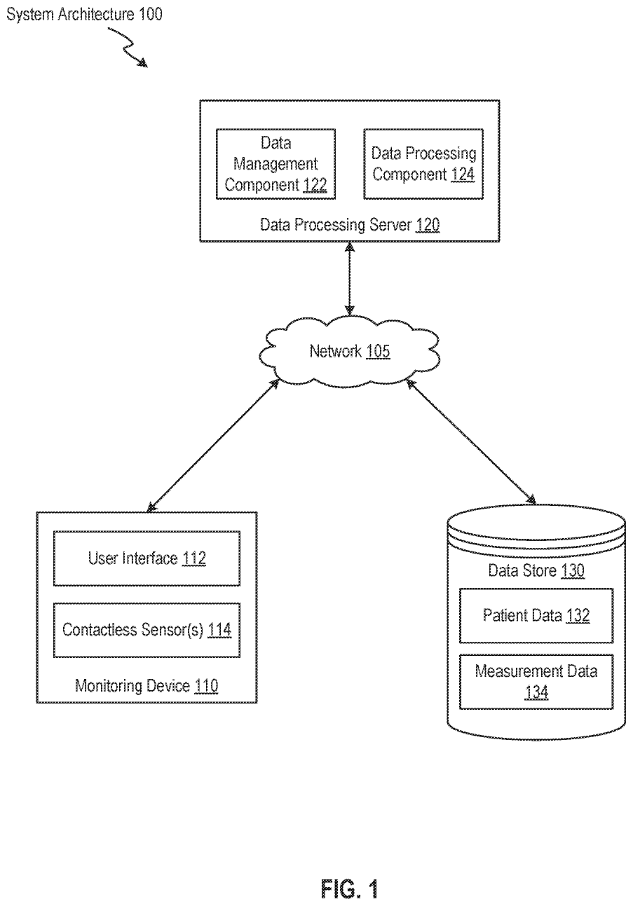

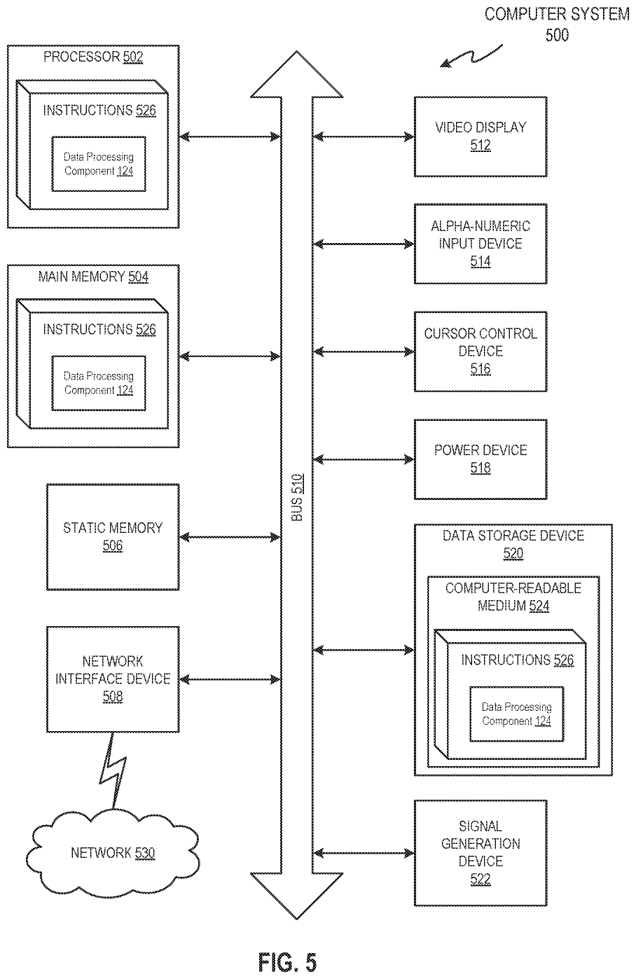

Exemplary implementations of the embodiments of the present disclosure are now described. FIG. 1 illustrates an exemplary system architecture 100 in accordance with at least one embodiment. The system architecture 100 includes a monitoring device 110, a data processing server 120, and a data store 130, with each device of the system architecture 100 being communicatively coupled via a network 105. One or more of the devices of the system architecture 100 may be implemented using a generalized computer system 500, described with respect to FIG. 5. The devices of the system architecture 100 are merely illustrative, and it is to be understood that other user devices, data processing servers, data stores, and networks may be present.

In one embodiment, network 105 may include a public network (e.g., the Internet), a private network (e.g., a local area network (LAN) or wide area network (WAN)), a wired network (e.g., Ethernet network), a wireless network (e.g., an 802.11 network or a Wi-Fi network), a cellular network (e.g., a Long Term Evolution (LTE) network), routers, hubs, switches, server computers, and/or a combination thereof. Although the network 105 is depicted as a single network, the network 105 may include one or more networks operating as stand-alone networks or in cooperation with each other. The network 105 may utilize one or more protocols of one or more devices to which they are communicatively coupled.

In one embodiment, the monitoring device 110 may include a computing device such as a personal computer (PC), laptop, mobile phone, smart phone, tablet computer, netbook computer, etc. The monitoring device 110 may also be a standalone device having one or more processing devices housed therein. An individual user may be associated with (e.g., own and/or operate) the monitoring device 110. As used herein, a “user” may be represented as a single individual. However, other embodiments of the present disclosure encompass a “user” being an entity controlled by a set of users and/or an automated source. For example, a set of individual users federated as a community in a company or government organization may be considered a “user.”

The monitoring device 110 may utilize one or more local data stores, which may be internal or external devices, and may each include one or more of a short-term memory (e.g., random access memory), a cache, a drive (e.g., a hard drive), a flash drive, a database system, or another type of component or device capable of storing data. The local data stores may also include multiple storage components (e.g., multiple drives or multiple databases) that may also span multiple computing devices (e.g., multiple server computers). In at least one embodiment, the local data stores may be used for data back-up or archival purposes.

The monitoring device 110 may implement a user interface 112, which may allow the monitoring device 110 to send/receive information to/from other monitoring devices (not shown), the data processing server 120, and the data store 130. The user interface 112 may be a graphical user interface (GUI). For example, the user interface 112 may be a web browser interface that can access, retrieve, present, and/or navigate content (e.g., web pages such as Hyper Text Markup Language (HTML) pages) provided by the data processing server 120. In one embodiment, the user interface 112 may be a standalone application (e.g., a mobile “app,” etc.), that enables a user to use the monitoring device 110 to send/receive information to/from other monitoring devices (not shown), the data processing server 120, and the data store 130. In at least one embodiment, the user interface 112 provides a visual readout of measurements or estimations of physiological parameters, such as blood volume change, made the monitoring device 110 or in combination with another device, and/or may provide instructions to the user operating the monitoring device 110 visually, aurally, or both.

In at least one embodiment, the monitoring device 110 comprises one or more contactless sensor(s) 114. The contactless sensor(s) 114 may include one or more high-resolution visible light cameras, infrared imaging sensors, motion radar imaging sensors, microphone array sensors, vibration sensors, accelerometer sensors, depth sensors, ambient temperature sensors, ambient humidity sensors, or other sensor devices. The contactless sensor(s) 114 may be at least partially disposed within a housing of the monitoring device 110 to protect the sensor.

In some embodiments, the contactless sensor(s) 114 include a high resolution visible light camera having a minimum resolution of 720p and frame rate of 30 fps (or up to 60 fps). In some embodiments, the contactless sensor(s) 114 include a long-wave infrared (LWIR) imaging sensor having a minimum resolution of 32 pixels to 128 pixels wide, and further enabled with radiometric and normalized sensing. In some embodiments, the contactless sensor(s) 114 include one or more 3D and motion radar imaging sensors using frequency-modulated continuous-wave (FMCW) or impulse radar technology at a short range. A first radar sensor may be configured to function in the 60 GHz-100 GHz band to provide high resolution images, but is not suited to penetrate typical home obstacles/barriers, such as walls or furniture (the “millimeter (mm) wave” radar, also referred to herein as “mmWave”). A second radar sensor may be configured to function in the 6-10 GHz band and is suited to penetrate most home obstacles/barriers at a range of about 10 m (ultra-wideband radar, also referred to herein as “UWB”). Both radar imaging sensors may also have onboard DSP to perform all the FMCW or pulse radar signal processing.

An RGB camera is mostly sensitive to the visible light wavelengths range (approximately from 400 to 1000 nm). Therefore, on a homogenous skin surface, color changes induced by blood flow variations can be measured by such a sensor. Starting from a live feed of RGB camera, a face detection algorithm can be performed in real time. The detected facial landmarks are used to extract regions of interest (ROIs) from the facial image.

In one embodiment, the data processing server 120 may include one or more computing devices (such as a rackmount server, a router computer, a server computer, a personal computer, a mainframe computer, a laptop computer, a tablet computer, a desktop computer, etc.), data stores (e.g., hard disks, memories, databases), networks, software components, and/or hardware components from which digital contents may be retrieved. In at least one embodiment, the data processing server 120 may be a server utilized by the monitoring device 110, for example, to process image data and provide the monitoring device 110 with resulting measurement or estimation data. In at least one embodiment, additional data processing servers may be present. In at least one embodiment, the data processing server 120 utilizes a data management component 122 to process, store, and analyze patient data 132 and measurement data 134 (which may be stored in the data store 130 or received directly from the monitoring device 110). In at least one embodiment, the data processing server further utilizes a data processing component 124 to process data obtained from contactless sensor(s) 114 and to compute measurements or estimates of one or more physiological parameters, such as blood volume change. The role of the data processing component 124 will be described in greater detail below.

In one embodiment, the data store 130 may include one or more of a short-term memory (e.g., random access memory), a cache, a drive (e.g., a hard drive), a flash drive, a database system, or another type of component or device capable of storing data. The data store 130 may also include multiple storage components (e.g., multiple drives or multiple databases) that may also span multiple computing devices (e.g., multiple server computers). In at least one embodiment, the data store 130 may be cloud-based. One or more of the devices of system architecture 100 may utilize their own storage and/or the data store 130 to store public and private data, and the data store 130 may be configured to provide secure storage for private data. In at least one embodiment, the data store 130 stores patient data 132, which may include health records of individual patients, biometric data, health conditions, and other information. In at least one embodiment, the data store 130 stores measurement data 134, which may include sensor data, vital signs, predictive data, alert data, or other information. The measurement data 134 may further include current data and historical data for training one or more machine learning models. Current data may be data (e.g., current sensor data, current vital signs, etc.) used to generate predictive data and/or used to re-train the one or more machine learning models. Each instance (e.g., set) of sensor data, vital signs, user information, predictive data, etc. may correspond to a respective user and/or a corresponding period of time.

Although each of the monitoring device 110, the data processing server 120, and the data store 130 are depicted in FIG. 1 as single, disparate components, these components may be implemented together in a single device or networked in various combinations of multiple different devices that operate together. In at least one embodiment, at least some of the functionality of the data processing server 120 and/or the data store 130 may be performed by the monitoring device 110 (e.g., a fully-functional standalone device), or by other devices.

Although embodiments of the disclosure are discussed in the context of rPPG measurements and estimates parameters such as blood volume change, such embodiments are generally applicable to other types of physiological measurements and to other areas of the body.

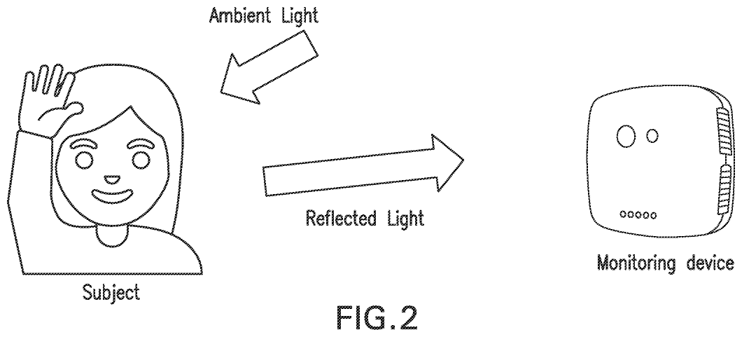

FIG. 2 illustrates a high level overview of the rPPG measurement performed by an exemplary device, in accordance with at least one embodiment. In at least one embodiment, the monitoring device (e.g., which may be representative of the monitoring device 110) captures ambient light reflected from the subject's face and/or the subject's hand. In at least one embodiment, the monitoring captures video of the subject's face and/or hand(s), from which one or more frames may be extracted and analyzed.

In at least one embodiment, at the start of a session, a subject can raise their hand or not. In the case where the individual has not raised their hand, if the algorithm determines that not enough light is reflected by the skin of the subject's face (e.g., due to the subject having a darker skin tone or facial obstruction, or low ambient light), an indication may be provided by the monitoring device (e.g., an audio indication, a visual indicator, or both) requesting that the user raise one or both hands while facing the sensor(s) of the monitoring device. Upon detection that the subject has complied (e.g., by detecting the presence of the subject's face and hand(s)), the monitoring device may begin to capture images for the purposes of measurement while the subject remains in the same position (e.g., for 2-10 seconds). In at least one embodiment, the monitoring device may further determine whether the subject is located at a suitable distance from the sensor(s) (e.g., a distance of about 1 meter) prior to commencing the image capture. In at least one embodiment, the monitoring device may further determine whether the amount of ambient light impinging on the subject is sufficient and does not saturate the images. In such cases, the monitoring device may provide an indication to the subject to adjust the lighting settings.

In at least one embodiment, the monitoring device utilizes an RGB camera, an infrared (IR) camera, a radar sensor, a combination thereof to capture images of the subjects face and/or hand(s). On a homogenous skin surface, color changes induced by blood flow variations can be measured by one or more of these sensors.

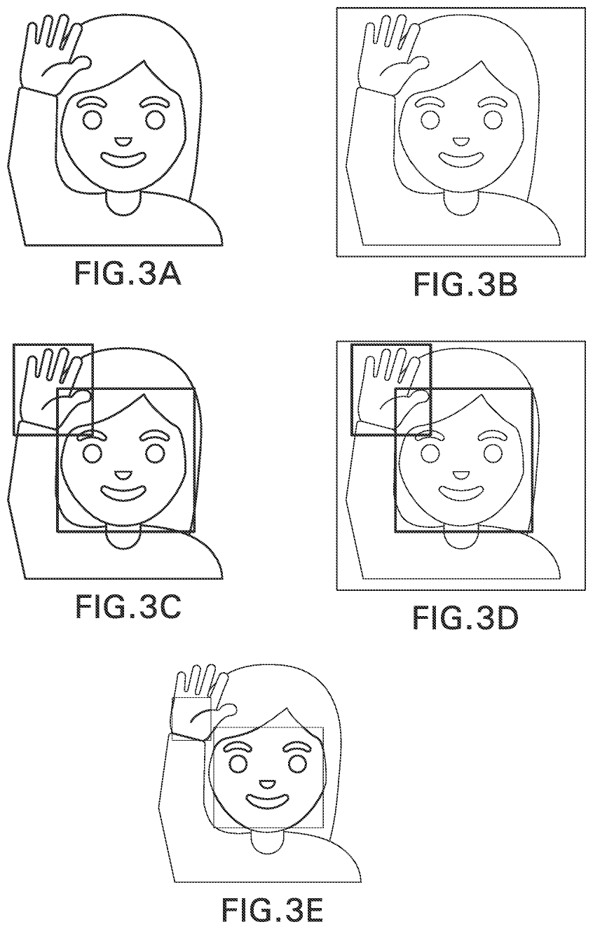

FIG. 3 illustrates face and hand detection for isolating regions of interest, in accordance with at least one embodiment. In at least one embodiment, both visible light camera images (FIG. 3A) and infrared camera images (FIG. 3B) are involved in blood volume change estimation. In at least one embodiment, a processing device (e.g., of the monitoring device) automatically detects the hand and face of the subject in both the visible light and infrared images, as illustrated by the bounding boxes. In at least one embodiment, starting from a live feed of RGB camera, a face detection algorithm is performed in real time.

In at least one embodiment, raw frames from the visible light camera are directly processed by bypassing the camera's image signal processor (ISP). In at least one embodiment, one or more performance settings associated with the ISP of the camera are disabled, including bad pixel correction, Bayer domain hardware noise reduction, high-order lens-shading compensation, auto-white-balance, de-mosaic filtering, 3×3 color transformation, color artifact suppression, downscaling, or edge enhancement. In at least one embodiment, two or more, three or more, four or more, five or more, or all of the aforementioned performance settings are disabled. In such embodiments, the acquired pixel values may be closer to the true photon count compared to the results produced by the default ISP performance settings. The true photon count may be representative of changes of reflectance of the skin innervated by the capillary vessels indicating the proportion of hemoglobin proteins in the blood. Analyzing the raw frames allows for the RGB camera to more accurately act as a measurement device, and therefore avoids implementing algorithms to compensate for the camera's intrinsic correction processing, as in smartphone-based applications. In at least one embodiment, the raw-RGB color space of a visible light camera image is first converted into the standard RGB color space by applying standard processing, such as white balancing and demosaicing.

In at least one embodiment, the subject's face and/or hand(s) are automatically detected on all the frames of the visible light camera images (FIG. 3C) and infrared camera images (FIG. 3D). Along with this detection, characteristic landmark points can be extracted. Such landmark points can include, for example, eyes, the nose, and the mouse for the face, and fingers and the palm for the hand. In at least one embodiment, the subject's skin areas are segmented and extracted (e.g., based at least partially on the extracted landmarks) combining the detection in the visible light and infrared images into a composite image (FIG. 3E). The identified skin areas can be compartmented into smaller areas, using either landmark-independent fixed-size regions of interest or landmark-based regions of interest. The size of a region of interest may be selected to be small enough to encompass a physiologically relevant part of the microvascular bed of tissue, but large enough to exhibit sufficient pixel intensity regularity. Pixels in each region of interest can then be statistically analyzed. In at least one embodiment, a set of criteria on pixel statistics is predefined such that regions that do not satisfy these criteria are discarded.

In at least one embodiment, the subject's relative pose with respect to the monitoring device is estimated by applying one or more computer vision algorithms to the visible light images, as well as by analyzing point cloud data provided by the radar sensor. Pose estimation can be used to improve the relevant image quality.

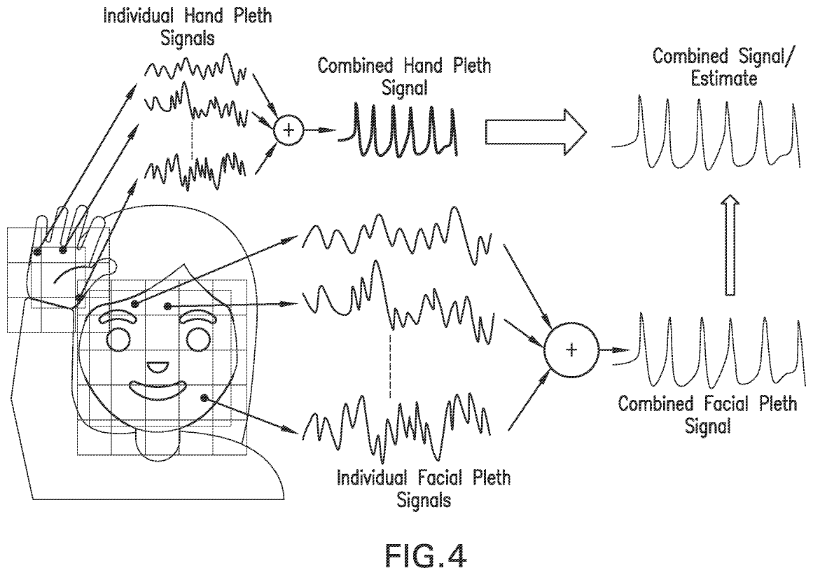

FIG. 4 illustrates generating of a composite pleth signal computed from signals derived from the subject's hand and face, in accordance with at least one embodiment. In at least one embodiment, after regions of interest in the images identified as skin are compartmentalized/segmented, a signal is extracted for each region of interest corresponding to the hand or face by processing pixel values. In at least one embodiment, multiple individual signals are combined and processed to reduce noise, resulting in a smoother periodic signal reflecting blood volume change dynamics. In at least one embodiment, signal quality is assessed by a confidence score (represented in FIG. 4 by the trace thickness) for both on the facial and the hand pleth signals. For example, each stream of processing (i.e., processing facial images/ROIs and processing hand images/ROIs) results in pleth signal and a time-dependent confidence score. Each confidence score can be determined by multiple factors, including both external factors (e.g., amount of facial movement) and internal factors (e.g., pleth regularity per pulse). For each stream (face or hand), several heart rates can be computed based on the outputted pleth signal, along with their associated confidence score. In at least one embodiment, a peak detection algorithm is used where the confidence score corresponds to the proportion of the signal covered by valid pulses. In at least one embodiment, a fast Fourier transform is used where the confidence score is computed as the signal-to-noise ratio. In at least one embodiment, a machine learning classifier is applied to the predictions, which include the multiple computed heart rates and their confidence scores over a short time window, both for the face and the hand. This approach can implement a voting mechanism that allows for the selection of the most reliable heart rate prediction, whether it is from the face or the hand. In at least one embodiment, after a confidence score is assessed for a facial pleth signal, it may be determined whether the confidence score satisfies a threshold condition (e.g., the confidence score is greater than or equal to a predefined value). If the confidence score fails to satisfy the threshold condition, a hand pleth signal is then obtained and used together with the facial pleth signal to perform estimates of vital signs. Otherwise, the system may proceed with just the facial pleth signal for performing estimates of vital signs.

In at least one embodiment, a hand pleth signal is captured regardless of whether the confidence score computed for the facial pleth signal satisfies the threshold condition. In such embodiments, a global confidence score is computed based on the highest confidence scores computed for the hand pleth and facial pleth signals, and the quality of the signals can be assessed based on whether the global confidence score satisfies the threshold condition.

Estimates of blood volume change may be computed for each region of interest in the segmented images. In at least one embodiment, bandpass filtering is applied to the signal to remove the lower frequency contribution of the body movement and to clean the higher frequency artifacts resulting from electronic noise and digital processing. The final estimates can then be combined from the facial and the hand estimates, weighted by their respective confidence scores.

In at least one embodiment, to estimate heart rate, all the extracted pleth signals are combined into a single composite pleth signal by weight averaging for face and hand separately. Other vital signs may be computed in a similar manner, such as, but not limited to, heart rate variability, respiratory rate, oxygen saturation, respiratory rate, and blood pressure.

In at least one embodiment, pulse transit time may be computed from pleth signals computed from the facial and hand images. In at least one embodiment, pulse transit time may be used to predict vital signs, such as blood pressure. Pulse transit time is defined as the time it takes for a pulse wave to travel between two arterial sites (e.g., the hand and the face, in the present embodiments). The time can be estimated by comparing the pleth of a small region on the hand and the pleth of a small region on the face. The choice of these regions can be determined by the quality of the two pleths individually and of their cross-correlation. In at least one embodiment, a confidence score quantifying the shapes of the matching pulses is used to weigh the contribution of each measured pulse transit time.

Upon processing images, various vital signs can be calculated. The vital signs may be stored to compile a profile of historical measurements for an individual. The stored vital signs data can be retrieved, for example, by a healthcare professional. In addition to being useful for computing various vital signs, the pleth data is medically valuable information in itself that can be used to qualitatively assess the general cardiovascular or respiratory health of a patient.

It is envisioned that the foregoing monitoring may be deployed in various settings. In one embodiment, the monitoring device may be deployed in an individual's home setting, wherein the vital signs measurements may be conducted on preconfigured schedule. For example, the monitoring device may be configured to notify the individual when it is time to measure the various vital signs as part of a daily monitoring routine for assessing heart health, prompting the individual to stand in close proximity to the monitoring device for sensor data acquisition. In another embodiment, the monitoring device may be configured for deployment in an environment where an individual is in a fairly stationary position. For example, the monitoring device may be implemented in a car dashboard to monitor driver health, above hospital beds, a self-service health checkup station, or any other applicable health monitoring setting.

One or more of the methods described herein may be performed by processing logic that may include hardware (e.g., circuitry, dedicated logic, programmable logic, microcode, processing device, etc.), software (such as instructions run on a processing device, a general purpose computer system, or a dedicated machine), firmware, microcode, or a combination thereof. In some embodiments, methods may be performed, in part, by the monitoring device 110 or the data processing server 120. In some embodiments, a non-transitory storage medium stores instructions that when executed by a processing device (e.g., of the monitoring device 110 or the data processing server 120) cause the processing device to perform methods.

For simplicity of explanation, the methods described herein are characterized as a series of acts or operations. However, acts in accordance with this disclosure can occur in various orders and/or concurrently and with other acts or operations not presented and described herein. Furthermore, not all illustrated acts or operations may be performed to implement the methods in accordance with the disclosed subject matter. In addition, those skilled in the art will understand and appreciate that the methods could alternatively be represented as a series of interrelated states via a state diagram or events.

FIG. 5 illustrates a diagrammatic representation of a machine in the exemplary form of a computer system 500 within which a set of instructions (e.g., for causing the machine to perform any one or more of the methodologies discussed herein) may be executed. In alternative embodiments, the machine may be connected (e.g., networked) to other machines in a LAN, an intranet, an extranet, or the Internet. The machine may operate in the capacity of a server or a client machine in client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Some or all of the components of the computer system 500 may be utilized by or illustrative of at least some of the devices of the system architecture 100, such as the monitoring device 110, the data processing server 120, and the data store 130.

The exemplary computer system 500 includes a processing device (processor) 502, a main memory 504 (e.g., read-only memory (ROM), flash memory, dynamic random access memory (DRAM) such as synchronous DRAM (SDRAM) or Rambus DRAM (RDRAM), etc.), a static memory 506 (e.g., flash memory, static random access memory (SRAM), etc.), and a data storage device 520, which communicate with each other via a bus 510.

Processor 502 represents one or more general-purpose processing devices such as a microprocessor, central processing unit, or the like. More particularly, the processor 502 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 502 may also be one or more special-purpose processing devices such as an ASIC, a field programmable gate array (FPGA), a digital signal processor (DSP), network processor, or the like. The processor 502 is configured to execute instructions 526 for performing the operations and steps discussed herein, such as operations associated with the data management component 122 or the data processing component 124.

The computer system 500 may further include a network interface device 508. The computer system 500 also may include a video display unit 512 (e.g., a liquid crystal display (LCD), a cathode ray tube (CRT), or a touch screen), an alphanumeric input device 514 (e.g., a keyboard), a cursor control device 516 (e.g., a mouse), and/or a signal generation device 522 (e.g., a speaker).

Power device 518 may monitor a power level of a battery used to power the computer system 500 or one or more of its components. The power device 518 may provide one or more interfaces to provide an indication of a power level, a time window remaining prior to shutdown of computer system 500 or one or more of its components, a power consumption rate, an indicator of whether computer system is utilizing an external power source or battery power, and other power related information. In at least one embodiment, indications related to the power device 518 may be accessible remotely (e.g., accessible to a remote back-up management module via a network connection). In at least one embodiment, a battery utilized by the power device 518 may be an uninterruptable power supply (UPS) local to or remote from computer system 500. In such embodiments, the power device 518 may provide information about a power level of the UPS.

The data storage device 520 may include a computer-readable storage medium 524 on which is stored one or more sets of instructions 526 (e.g., software) embodying any one or more of the methodologies or functions described herein. The instructions 526 may also reside, completely or at least partially, within the main memory 504 and/or within the processor 502 during execution thereof by the computer system 500, the main memory 504 and the processor 502 also constituting computer-readable storage media. The instructions 526 may further be transmitted or received over a network 530 (e.g., the network 105) via the network interface device 508.

In one embodiment, the instructions 526 include instructions for implementing the functionality of the data processing server 120, as described throughout this disclosure. While the computer-readable storage medium 524 is shown in an exemplary embodiment to be a single medium, the terms “computer-readable storage medium” or “machine-readable storage medium” should be taken to include a single medium or multiple media (e.g., a centralized or distributed database, and/or associated caches and servers) that store the one or more sets of instructions. The terms “computer-readable storage medium” or “machine-readable storage medium” shall also be taken to include any transitory or non-transitory medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “receiving,” “determining,” “providing,” “combining,” “training,” “obtaining,” “identifying,” “computing,” “estimating,” “capturing,” “generating,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

In the above description, numerous details are set forth. It will be apparent, however, to one of ordinary skill in the art having the benefit of this disclosure, that embodiments may be practiced without these specific details. In some instances, well-known structures and devices are shown in block diagram form, rather than in detail, in order to avoid obscuring the description.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

Embodiments also relate to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, such as, but not limited to, any type of disk including floppy disks, optical disks, CD-ROMs and magnetic-optical disks, read-only memories (ROMs), random access memories (RAMs), EPROMS, EEPROMs, magnetic or optical cards, or any type of media suitable for storing electronic instructions.

The algorithms, methods, and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description above. In addition, the present embodiments are not described with reference to any particular programming language. It will be appreciated that a variety of programming languages may be used to implement the teachings of the present invention as described herein. It should also be noted that the terms “when” or the phrase “in response to,” as used herein, should be understood to indicate that there may be intervening time, intervening events, or both before the identified operation is performed.

Various operations are described as multiple discrete operations, in turn, in a manner that is most helpful in understanding the present disclosure, however, the order of description should not be construed to imply that these operations are necessarily order dependent. In particular, these operations need not be performed in the order of presentation. The following exemplary embodiments are now described:

Embodiment 1: A method of assessing at least one vital sign of a subject, the method comprising: acquiring a first pleth signal based at least in part on capturing ambient light reflected from at least one region of interest (ROI) on a face of the subject; computing a confidence score for the first pleth signal; acquiring a second pleth signal from a palmar side of a hand of the subject, wherein the second pleth signal is acquired when the confidence score in the first signal does not satisfy a threshold value; and computing estimates of the at least one vital sign of the subject from one or both of the first and second pleth signals and combining the estimates into a final estimate of the at least one vital sign based on the confidence score.

Embodiment 2: A method of assessing at least one vital sign of a subject, the method comprising: acquiring a first pleth signal based at least in part on capturing ambient light reflected from at least one region of interest (ROI) on a face of the subject; computing a confidence score for the first pleth signal; acquiring a second pleth signal from a palmar side of a hand of the subject, wherein the second pleth signal is acquired when the confidence score in the first signal does not satisfy a threshold value; combining the first and second pleth signals into a composite pleth signal based on the confidence score; and estimating the at least one vital sign of the subject from the composite pleth signal.

Embodiment 3: A method of assessing at least one vital sign of a subject, the method comprising: acquiring a first pleth signal based at least in part on capturing ambient light reflected from at least one region of interest (ROI) on a face of the subject; computing a confidence score for the first pleth signal; determining whether the confidence score satisfies a threshold condition; and responsive to determining that the confidence score fails to satisfy the threshold condition, acquiring a second pleth signal from a palmar side of a hand of the subject.

Embodiment 4: A method of assessing at least one vital sign of a subject, the method comprising: acquiring a first pleth signal based at least in part on capturing ambient light reflected from at least one region of interest (ROI) on a face of the subject; acquiring a second pleth signal from a palmar side of a hand of the subject; computing a confidence score for the first pleth signal or the second pleth signal; and computing estimates of the at least one vital sign of the subject from the first and second pleth signals and combining the estimates into a final estimate of the at least one vital sign based on the confidence score.

Embodiment 5: The method of any of Embodiments 1-4, wherein the confidence score is computed based on one or more of ambient light, movement of the subject, and regularity of the first pleth signal.

Embodiment 6: The method of any of Embodiments 1-5, further comprising: computing a pulse transmit time based at least in part on the first pleth signal and the second pleth signal.

Embodiment 7: The method of any of Embodiments 1-6, wherein the subject is prompted to position the palmar side of the hand for acquisition of the second pleth signal.

Embodiment 8: The method of any of Embodiments 1-7, further comprising determining a second confidence score in the second pleth signal.

Embodiment 9: The method of Embodiment 8, wherein the first pleth signal and the second pleth signal or their respective estimates are weighted by their respective confidence scores.

Embodiment 10: The method of any of Embodiments 1-9, wherein the first pleth signal is derived from sub-signals collected from a plurality of ROIs on the face of the subject.

Embodiment 11: The method of any of Embodiments 1-10, wherein the second pleth signal is derived from sub-signals collected from a plurality of ROIs on the hand of the subject.

Embodiment 12: The method of any of Embodiments 1-11, wherein the first and second pleth signals are derived from image frames collected using a visible light camera and an infrared camera, the infrared camera configured to extract data associated with skin areas inside ROIs identified on image frames collected by the visible light camera.

Embodiment 13: The method of any of Embodiments 1-12, wherein the vital sign of the subject assessed is a heart rate, a variability in hearth rate, a respiratory rate, an oxygen saturation, a blood pressure or a combination thereof.

Embodiment 14: A device to perform remote photoplethysmography on a subject, the device comprising: a camera; and a processing device communicatively coupled to camera, wherein the processing device is configured to perform any of the methods of Embodiments 1-13.

Embodiment 15: A system comprising: a memory; and a processing device communicatively coupled to the memory, wherein the processing device is configured to perform any of the methods of Embodiments 1-13.

Embodiment 16: A non-transitory computer-readable medium having instructions stored thereon, which, when executed by a processing device, cause the processing device to perform the method of any of Embodiments 1-13.

While the present disclosure has been described with respect to a limited number of embodiments, those skilled in the art will appreciate numerous modifications and variations therefrom. It is intended that the appended claims cover all such modifications and variations as fall within the true spirit and scope of this present disclosure.

Use of the phrase “configured to” refers to an apparatus, hardware, logic, or other element that is adapted, arrange, programmed, or otherwise capable of performing a designated or determined task by itself or in combination with additional apparatuses, hardware, logic, or other elements. For example, an apparatus or element thereof that is not operating is still “configured to” perform a designated task if it is designed, coupled, and/or interconnected to perform said designated task.

In the foregoing specification, a detailed description has been given with reference to specific exemplary embodiments. It will, however, be evident that various modifications and changes can be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment and other exemplarily language does not necessarily refer to the same embodiment or the same example, but can refer to different and distinct embodiments, as well as potentially the same embodiment.

The words “example” or “exemplary” are used herein to mean serving as an example, instance or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

What is claimed is:

1. A method of assessing at least one vital sign of a subject, the method comprising:
acquiring a first pleth signal based at least in part on capturing ambient light reflected from at least one region of interest (ROI) on a face of the subject;
acquiring a second pleth signal from a palmar side of a hand of the subject;
computing a confidence score for the first pleth signal or the second pleth signal; and
computing estimates of the at least one vital sign of the subject from the first and second pleth signals and combining the estimates into a final estimate of the at least one vital sign based on the confidence score.

2. The method of claim 1, wherein the confidence score is computed based on one or more of ambient light, movement of the subject, and regularity of the first pleth signal.

3. The method of claim 1, further comprising:
computing a pulse transmit time based at least in part on the first pleth signal and the second pleth signal.

4. The method of claim 1, wherein the subject is prompted to position the palmar side of the hand for acquisition of the second pleth signal.

5. The method of claim 1, further comprising determining a second confidence score in the second pleth signal.

6. The method of claim 5, wherein the first pleth signal and the second pleth signal or their respective estimates are weighted by their respective confidence scores.

7. The method of claim 1, wherein the first pleth signal is derived from sub-signals collected from a plurality of ROIs on the face of the subject.

8. The method of claim 1, wherein the second pleth signal is derived from sub-signals collected from a plurality of ROIs on the hand of the subject.

9. The method of claim 1, wherein the first and second pleth signals are derived from image frames collected using a visible light camera and an infrared camera, the infrared camera configured to extract data associated with skin areas inside ROIs identified on image frames collected by the visible light camera.

10. The method of claim 1, wherein the vital sign of the subject assessed is a heart rate, a variability in hearth rate, a respiratory rate, an oxygen saturation, a blood pressure or a combination thereof.

11. A device to perform remote photoplethysmography on a subject, the device comprising:
a camera; and
a processing device communicatively coupled to camera, wherein the processing device is configured to:
acquire a first pleth signal based at least in part on capturing ambient light reflected from at least one region of interest (ROI) on a face of the subject;
acquire a second pleth signal from a palmar side of a hand of the subject;
compute a confidence score for the first pleth signal or the second pleth signal; and
compute estimates of at least one vital sign of the subject from the first and second pleth signals and combining the estimates into a final estimate of at least one vital sign based on the confidence score.

12. The device of claim 11, wherein the confidence score is computed based on one or more of ambient light, movement of the subject, and regularity of the first pleth signal.

13. The device of claim 11, wherein the processing device is further configured to:
compute a pulse transmit time based at least in part on the first pleth signal and the second pleth signal.

14. The device of claim 11, wherein the subject is prompted to position the palmar side of the hand for acquisition of the second pleth signal.

15. The device of claim 11, wherein the processing device is further configured to:
determine a second confidence score in the second pleth signal, wherein the first pleth signal and the second pleth signal or their respective estimates are weighted by their respective confidence scores.

16. The device of claim 11, wherein the first pleth signal is derived from sub-signals collected from a plurality of ROIs on the face of the subject.

17. The device of claim 11, wherein the second pleth signal is derived from sub-signals collected from a plurality of ROIs on the hand of the subject.

18. The device of claim 11, wherein the first and second pleth signals are derived from image frames collected using a visible light camera and an infrared camera, the infrared camera configured to extract data associated with skin areas inside ROIs identified on image frames collected by the visible light camera.

19. The device of claim 11, wherein the at least one vital sign of the subject assessed is a heart rate, a variability in hearth rate, a respiratory rate, an oxygen saturation, a blood pressure or a combination thereof.

20. A non-transitory computer-readable medium having instructions stored thereon, which, when executed by a processing device, cause the processing device to:
acquire a first pleth signal based at least in part on capturing ambient light reflected from at least one region of interest (ROI) on a face of a subject;
acquire a second pleth signal from a palmar side of a hand of the subject;
compute a confidence score for the first pleth signal or the second pleth signal; and
compute estimates of the at least one vital sign of the subject from the first and second pleth signals and combining the estimates into a final estimate of the at least one vital sign based on the confidence score.
